(19) United States
(12) Patent Application Publication
(10) Pub. No.: US 2026/0356821 A1
(43) Pub. Date: Nov. 12, 2026

(54) SOLID FORMS OF AN ERBB INHIBITOR

(71) Applicant: Black Diamond Therapeutics, Inc., Cambridge, MA (US)

(72) Inventors: Santosh KUMAR, Lexington, MA (US); Kristos Adrian MOSHOS, Belmont, MA (US); Wu-Yan ZHANG, Lexington, MA (US); Reza FOROUGHI, Pacific Palisades, CA (US)

(21) Appl. No.: 19/712,749

(22) Filed: Jun. 18, 2026

Related U.S. Application Data
(63) Continuation of application No. PCT/US2024/061246, filed on Dec. 20, 2024.
(60) Provisional application No. 63/613,537, filed on Dec. 21, 2023.

(51) Int. Cl. C07D 403/06; A61K 31/5377

(52) U.S. Cl. CPC C07D 403/06; A61K 31/5377

(57) ABSTRACT

The present disclosure provides a morphic form of a pharmaceutically acceptable salt of Compound No. 1:

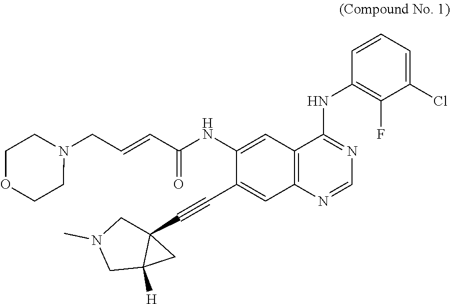 [SMILES: CN1C[C@H]2C[C@@]2(C#Cc2cc3ncnc(Nc4cccc(Cl)c4F)c3cc2NC(=O)/C=C/CN2CCOCC2)C1]

solvates thereof and hydrates thereof. The present disclosure also provides processes for preparing the morphic form and uses of the morphic form, e.g., in preventing or treating cancer.

### RELATED APPLICATIONS

This application is a continuation of International Application No. PCT/US2024/061246, filed on Dec. 20, 2024, which claims priority to, and the benefit of, U.S. Provisional Application No. 63/613,537 filed Dec. 21, 2023, the entire contents of each of which are hereby incorporated by reference for all purposes.

### BACKGROUND

Mutations affecting either the intracellular catalytic domain or extracellular ligand binding domain of an ErbB receptor can generate oncogenic activity (the ErbB protein family consists of 4 members including ErbB-1, also named epidermal growth factor receptor (EGFR) and Erb-2, also named HER2 in humans). ErbB inhibitors are a known treatment for a number of cancers. However, not every patient is responsive satisfactorily to this treatment. Thus, there is a long-felt need in the art for new therapies that are able to address the variable responsiveness of cancer patients to known therapies. The present disclosure provides compositions and methods for preventing or treating cancer in patients with these oncogenic mutations.

### SUMMARY

In some aspects, the present disclosure provides a morphic form of Compound No. 1:

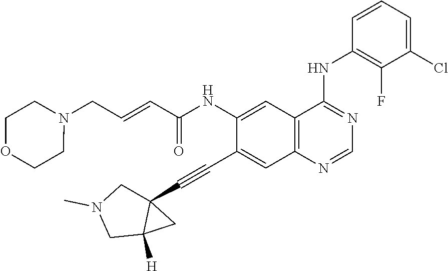 [SMILES: CN1C[C@H]2C[C@@]2(C#Cc2cc3ncnc(Nc4cccc(Cl)c4F)c3cc2NC(=O)/C=C/CN2CCOCC2)C1]

a solvate thereof, a hydrate thereof, or a pharmaceutically acceptable salt thereof.

In some aspects, the present disclosure provides a pharmaceutical composition comprising a therapeutically effective amount of a morphic form of Compound No. 1, a solvate thereof, a hydrate thereof, or a pharmaceutically acceptable salt thereof, and a pharmaceutically acceptable excipient.

In some aspects, the present disclosure provides a method of inhibiting an oncogenic variant of an ErbB receptor, comprising administering to the subject in need thereof a therapeutically effective amount of a morphic form of Compound No. 1, a solvate thereof, a hydrate thereof, or a pharmaceutically acceptable salt thereof.

In some aspects, the present disclosure provides a method of preventing or treating cancer, comprising administering to the subject in need thereof a therapeutically effective amount of a morphic form of Compound No. 1, a solvate thereof, a hydrate thereof, or a pharmaceutically acceptable salt thereof.

In some aspects, the present disclosure provides a morphic form of Compound No. 1, a solvate thereof, a hydrate thereof, or a pharmaceutically acceptable salt thereof, for use in the inhibition of an oncogenic variant of an ErbB receptor.

In some aspects, the present disclosure provides a morphic form of Compound No. 1, a solvate thereof, a hydrate thereof, or a pharmaceutically acceptable salt thereof, for use in the prevention or treatment of cancer.

In some aspects, the present disclosure provides use of a morphic form of Compound No. 1, a solvate thereof, a hydrate thereof, or a pharmaceutically acceptable salt thereof, in the manufacture of a medicament for inhibiting an oncogenic variant of an ErbB receptor.

In some aspects, the present disclosure provides use of a morphic form of Compound No. 1, a solvate thereof, a hydrate thereof, or a pharmaceutically acceptable salt thereof, in the manufacture of a medicament for preventing or treating cancer.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTIONS OF FIGURES

FIG. 1 depicts the XRPD pattern of Form M of Compound No. 1.

FIG. 2 depicts the DSC thermogram of Form M of Compound No. 1.

FIG. 3 depicts the TGA thermogram of Form M of Compound No. 1.

### DETAILED DESCRIPTION

It is understood that the term “Compound No. 1,” as used herein, refers to a compound having the following structure:

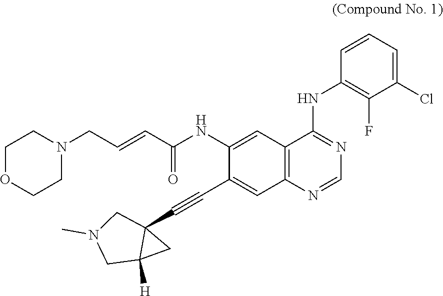 [SMILES: CN1C[C@H]2C[C@@]2(C#Cc2cc3ncnc(Nc4cccc(Cl)c4F)c3cc2NC(=O)/C=C/CN2CCOCC2)C1]

Compound No. 1 may be identified by the IUPAC name of (E)-N-(4-((3-chloro-2-fluorophenyl)amino)-7-(((1R,5S)-3-methyl-3-azabicyclo[3.1.0]hexan-1-yl)ethynyl)quinazolin-6-yl)-4-morpholinobut-2-enamide.

In some aspects, the present disclosure provides a morphic form of a pharmaceutically acceptable salt of Compound No. 1, a solvate thereof, or a hydrate thereof.

In some embodiments, the morphic form is a crystalline form.

In some embodiments, the morphic form is a morphic form (e.g., crystalline form) of a pharmaceutically acceptable salt of Compound No. 1.

In some embodiments, the morphic form (e.g., crystalline form) is Form M.

In some aspects, the present disclosure provides a method of preparing a morphic form of a pharmaceutically acceptable salt of Compound No. 1, a solvate thereof, or a hydrate thereof.

### Form M

### X-Ray Powder Diffraction (XRPD) Characterization

In some embodiments, the morphic form is Form M of a pharmaceutically acceptable salt of Compound No. 1, a solvate thereof, or a hydrate thereof.

In some embodiments, Form M is a pharmaceutically acceptable salt of Compound No. 1.

In some embodiments, Form M is a malate salt of Compound No. 1.

In some embodiments, Form M is an L-malate salt of Compound No. 1.

In some embodiments, Form M is a crystalline form.

In some embodiments, Form M is a crystalline form of a pharmaceutically acceptable salt of Compound No. 1.

In some embodiments, Form M is a crystalline form of a malate salt of Compound No. 1.

In some embodiments, Compound No. 1 and malate are present in Form M at a ratio of about 1:1.5, about 1:1.6, about 1:1.7, about 1:1.8, about 1:1.9, about 1:2.0, about 1:2.1, about 1:2.2, about 1:2.3, about 1:2.4, about 1:2.5, about 1:2.6, about 1:2.7, about 1:2.8, about 1:2.9, or about 1:3.0.

In some embodiments, Form M is a crystalline form of an L-malate salt of Compound No. 1.

In some embodiments, Form M is characterized by an X-ray powder diffraction (“XRPD”) pattern comprising signals (e.g., peaks) at 4.5±0.2, 8.9±0.2, and 21.7±0.2°2θ (e.g., 4.5±0.1, 8.9±0.1, and 21.7±0.1°2θ (e.g., 4.5, 8.9, and 21.7° 2θ)) using Cu Kα radiation.

In some embodiments, the XRPD pattern of Form M further comprises at least one signal (e.g., peak) selected from 13.3±0.2, 13.9±0.2, and 19.7±0.2°2θ (e.g., 13.3±0.1, 13.9±0.1, and 19.7±0.1°2θ (e.g., 13.3, 13.9, and 19.7° 2θ)) using Cu Kα radiation.

In some embodiments, the XRPD pattern of Form M further comprises at least two signals (e.g., peaks) selected from 13.3±0.2, 13.9±0.2, and 19.7±0.2°2θ (e.g., 13.3±0.1, 13.9±0.1, and 19.7±0.1°2θ (e.g., 13.3, 13.9, and 19.7° 2θ)) using Cu Kα radiation.

In some embodiments, the XRPD pattern of Form M further comprises signals (e.g., peaks) at 13.3±0.2, 13.9±0.2, and 19.7±0.2° 2θ (e.g., 13.3±0.1, 13.9±0.1, and 19.7±0.1° 2θ (e.g., 13.3, 13.9, and 19.7° 2θ)) using Cu Kα radiation.

In some embodiments, Form M is characterized by an X-ray powder diffraction (“XRPD”) pattern comprising at least three signals (e.g., peaks) selected from 4.5±0.2, 8.9±0.2, 13.3±0.2, 13.9±0.2, 19.7±0.2, and 21.7±0.2°2θ (e.g., 4.5±0.1, 8.9±0.1, 13.3±0.1, 13.9±0.1, 19.7±0.1, and 21.7±0.1°2θ (e.g., 4.5, 8.9, 13.3, 13.9, 19.7, and 21.7° 2θ)) using Cu Kα radiation.

In some embodiments, the XRPD pattern of Form M comprises at least four signals (e.g., peaks) selected from 4.5±0.2, 8.9±0.2, 13.3±0.2, 13.9±0.2, 19.7±0.2, and 21.7±0.2°2θ (e.g., 4.5±0.1, 8.9±0.1, 13.3±0.1, 13.9±0.1, 19.7±0.1, and 21.7±0.1°2θ (e.g., 4.5, 8.9, 13.3, 13.9, 19.7, and 21.7° 2θ)) using Cu Kα radiation.

In some embodiments, the XRPD pattern of Form M comprises at least five signals (e.g., peaks) selected from 4.5±0.2, 8.9±0.2, 13.3±0.2, 13.9±0.2, 19.7±0.2, and 21.7±0.2°2θ (e.g., 4.5±0.1, 8.9±0.1, 13.3±0.1, 13.9±0.1, 19.7±0.1, and 21.7±0.1°2θ (e.g., 4.5, 8.9, 13.3, 13.9, 19.7, and 21.7° 2θ)) using Cu Kα radiation.

In some embodiments, the XRPD pattern of Form M comprises signals (e.g., peaks) at 4.5±0.2, 8.9±0.2, 13.3±0.2, 13.9±0.2, 19.7±0.2, and 21.7±0.2° 2θ (e.g., 4.5±0.1, 8.9±0.1, 13.3±0.1, 13.9±0.1, 19.7±0.1, and 21.7±0.1° 2θ (e.g., 4.5, 8.9, 13.3, 13.9, 19.7, and 21.7° 2θ)) using Cu Kα radiation.

In some embodiments, the XRPD pattern of Form M further comprises at least one signal (e.g., peak) selected from 17.9±0.2, 18.9±0.2, 22.2±0.2, and 26.7±0.2°2θ (e.g., 17.9±0.1, 18.9±0.1, 22.2±0.1, and 26.7±0.1°2θ (e.g., 17.9, 18.9, 22.2, and 26.7° 2θ)) using Cu Kα radiation.

In some embodiments, the XRPD pattern of Form M further comprises at least two signals (e.g., peaks) selected from 17.9±0.2, 18.9±0.2, 22.2±0.2, and 26.7±0.2°2θ (e.g., 17.9±0.1, 18.9±0.1, 22.2±0.1, and 26.7±0.1°2θ (e.g. 17.9, 18.9, 22.2, and 26.7° 2θ)) using Cu Kα radiation.

In some embodiments, the XRPD pattern of Form M further comprises at least three signals (e.g., peaks) selected from 17.9±0.2, 18.9±0.2, 22.2±0.2, and 26.7±0.2°2θ (e.g., 17.9±0.1, 18.9±0.1, 22.2±0.1, and 26.7±0.1°2θ (e.g. 17.9, 18.9, 22.2, and 26.7° 2θ)) using Cu Kα radiation.

In some embodiments, the XRPD pattern of Form M comprises signals (e.g., peaks) at 4.5±0.2, 8.9±0.2, 13.3±0.2, 13.9±0.2, 17.9±0.2, 18.9±0.2, 19.7±0.2, 21.7±0.2, 22.2±0.2, and 26.7±0.2° 2θ (e.g. 4.5±0.1, 8.9±0.1, 13.3±0.1, 13.9±0.1, 17.9±0.1, 18.9±0.1, 19.7±0.1, 21.7±0.1, 22.2±0.1, and 26.7±0.1°2θ (e.g. 4.5, 8.9, 13.3, 13.9, 17.9, 18.9, 19.7, 21.7, 22.2, and 26.7° 2θ)) using Cu Kα radiation.

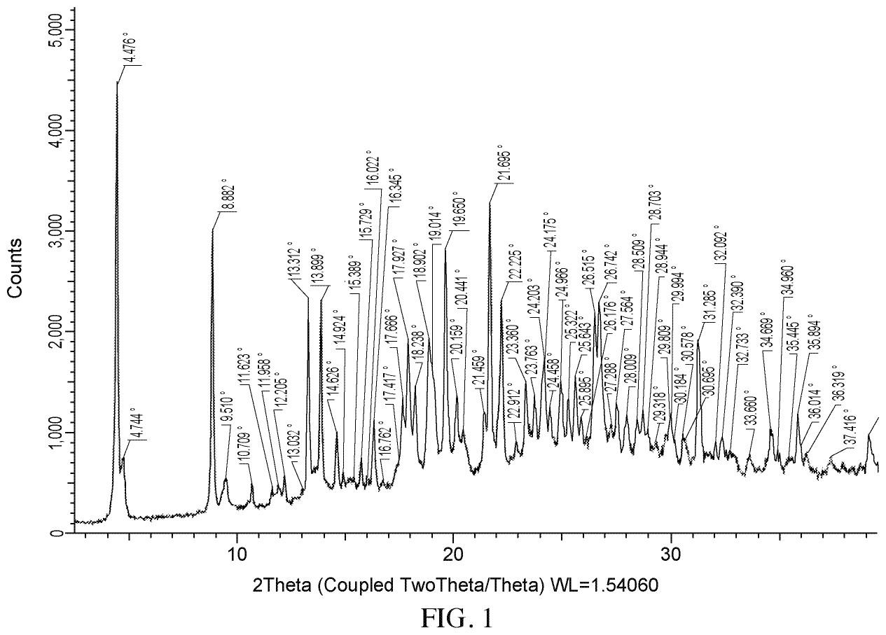

In some embodiments, Form M is characterized by an XRPD pattern substantially similar to that shown in FIG. 1.

In some embodiments, Form M is characterized by an XRPD pattern comprising three or more of the signals (e.g., peaks) at approximately the positions shown in Table A.

In some embodiments, Form M is characterized by an XRPD pattern comprising four or more of the signals (e.g., peaks) at approximately the positions shown in Table A.

In some embodiments, Form M is characterized by an XRPD pattern comprising five or more of the signals (e.g., peaks) at approximately the positions shown in Table A.

In some embodiments, Form Mis characterized by an XRPD pattern comprising six or more of the signals (e.g., peaks) at approximately the positions shown in Table A.

In some embodiments, Form M is characterized by an XRPD pattern comprising seven or more of the signals (e.g., peaks) at approximately the positions shown in Table A.

In some embodiments, Form M is characterized by an XRPD pattern comprising eight or more of the signals (e.g., peaks) at approximately the positions shown in Table A.

In some embodiments, Form M is characterized by an XRPD pattern comprising nine or more of the signals (e.g., peaks) at approximately the positions shown in Table A.

In some embodiments, Form M is characterized by an XRPD pattern comprising 10 or more of the signals (e.g., peaks) at approximately the positions shown in Table A.

In some embodiments, Form M is characterized by an XRPD pattern comprising signals (e.g., peaks) at approximately the positions shown in Table 1.

It is understood that the values in the table are approximate and subject to instrumental and experimental variations.

TABLE A
Exemplary XRPD Signal List for Form M

| | d | | Net | Gross | Rel. |
|---|---|---|---|---|---|
| Angle | Value | | Intensity | Intensity | Intensity |
| 4.476° | 19.72531 | Å | 4309.72 | 4443.73 | 100.0% |
| 4.744° | 18.61093 | Å | 591.152 | 729.635 | 13.7% |
| 8.882° | 9.94748 | Å | 2772.63 | 2994.30 | 64.3% |
| 9.510° | 9.29252 | Å | 300.936 | 542.050 | 7.0% |
| 10.709° | 8.25432 | Å | 231.183 | 496.675 | 5.4% |
| 11.623° | 7.60736 | Å | 154.130 | 443.966 | 3.6% |
| 11.958° | 7.39520 | Å | 151.657 | 454.744 | 3.5% |
| 12.205° | 7.24625 | Å | 256.575 | 568.468 | 6.0% |
| 13.032° | 6.78818 | Å | 113.907 | 462.952 | 2.6% |
| 13.312° | 6.64569 | Å | 1962.95 | 2325.38 | 45.5% |
| 13.899° | 6.36624 | Å | 1918.03 | 2305.03 | 44.5% |
| 14.626° | 6.05147 | Å | 594.453 | 1005.48 | 13.8% |
| 14.924° | 5.93120 | Å | 178.750 | 597.580 | 4.1% |
| 15.389° | 5.75302 | Å | 102.991 | 531.613 | 2.4% |
| 15.729° | 5.62962 | Å | 265.765 | 699.705 | 6.2% |
| 16.022° | 5.52740 | Å | 129.640 | 566.926 | 3.0% |
| 16.345° | 5.41875 | Å | 670.937 | 1110.58 | 15.6% |
| 16.762° | 5.28480 | Å | 70.4934 | 519.645 | 1.6% |
| 17.417° | 5.08756 | Å | 181.744 | 667.583 | 4.2% |
| 17.666° | 5.01649 | Å | 826.035 | 1326.84 | 19.2% |
| 17.927° | 4.94396 | Å | 1392.23 | 1907.86 | 32.3% |
| 18.238° | 4.86035 | Å | 913.003 | 1445.10 | 21.2% |
| 18.902° | 4.69116 | Å | 1362.05 | 1924.94 | 31.6% |
| 19.014° | 4.66364 | Å | 1082.97 | 1650.50 | 25.1% |
| 19.650° | 4.51428 | Å | 2226.78 | 2817.29 | 51.7% |
| 20.159° | 4.40138 | Å | 732.924 | 1337.94 | 17.0% |
| 20.441° | 4.34136 | Å | 394.427 | 1005.98 | 9.2% |
| 21.459° | 4.13755 | Å | 587.626 | 1236.50 | 13.6% |
| 21.695° | 4.09311 | Å | 2610.62 | 3273.31 | 60.6% |
| 22.225° | 3.99659 | Å | 1607.64 | 2298.71 | 37.3% |
| 22.912° | 3.87839 | Å | 186.490 | 911.371 | 4.3% |
| 23.360° | 3.80497 | Å | 737.159 | 1482.73 | 17.1% |
| 23.763° | 3.74136 | Å | 572.898 | 1334.76 | 13.3% |
| 24.175° | 3.67852 | Å | 873.005 | 1649.28 | 20.3% |
| 24.203° | 3.67434 | Å | 991.426 | 1768.60 | 23.0% |
| 24.458° | 3.63652 | Å | 459.244 | 1244.12 | 10.7% |
| 24.966° | 3.56369 | Å | 703.610 | 1501.18 | 16.3% |
| 25.322° | 3.51450 | Å | 489.911 | 1294.32 | 11.4% |
| 25.643° | 3.47114 | Å | 746.906 | 1556.04 | 17.3% |
| 25.895° | 3.43800 | Å | 340.560 | 1152.42 | 7.9% |
| 26.176° | 3.40172 | Å | 141.109 | 955.015 | 3.3% |
| 26.515° | 3.35889 | Å | 1333.18 | 2148.14 | 30.9% |
| 26.742° | 3.33096 | Å | 1473.43 | 2288.24 | 34.2% |
| 27.288° | 3.26548 | Å | 234.316 | 1045.93 | 5.4% |
| 27.564° | 3.23348 | Å | 462.306 | 1270.78 | 10.7% |
| 28.009° | 3.18303 | Å | 326.346 | 1127.60 | 7.6% |
| 28.509° | 3.12834 | Å | 320.624 | 1110.61 | 7.4% |
| 28.703° | 3.10769 | Å | 416.773 | 1201.50 | 9.7% |
| 28.944° | 3.08233 | Å | 249.394 | 1026.85 | 5.8% |
| 29.318° | 3.04392 | Å | 154.113 | 918.795 | 3.6% |
| 29.809° | 2.99484 | Å | 225.333 | 970.344 | 5.2% |
| 29.994° | 2.97683 | Å | 384.468 | 1121.25 | 8.9% |
| 30.184° | 2.95852 | Å | 178.905 | 906.750 | 4.2% |
| 30.578° | 2.92126 | Å | 292.597 | 1005.21 | 6.8% |
| 30.695° | 2.91042 | Å | 244.037 | 953.121 | 5.7% |
| 31.285° | 2.85684 | Å | 1201.66 | 1892.43 | 27.9% |
| 32.092° | 2.78685 | Å | 239.002 | 899.280 | 5.5% |
| 32.390° | 2.76182 | Å | 282.348 | 929.111 | 6.6% |
| 32.733° | 2.73372 | Å | 175.452 | 805.269 | 4.1% |
| 33.660° | 2.66048 | Å | 181.408 | 781.933 | 4.2% |
| 34.669° | 2.58533 | Å | 413.340 | 1019.06 | 9.6% |
| 34.960° | 2.56449 | Å | 206.472 | 811.152 | 4.8% |
| 35.445° | 2.53051 | Å | 148.433 | 748.860 | 3.4% |
| 35.894° | 2.49983 | Å | 577.744 | 1171.41 | 13.4% |
| 36.014° | 2.49179 | Å | 279.585 | 871.000 | 6.5% |
| 36.319° | 2.47159 | Å | 215.089 | 799.893 | 5.0% |
| 37.416° | 2.40158 | Å | 174.498 | 758.373 | 4.0% |
| 39.176° | 2.29766 | Å | 358.231 | 970.021 | 8.3% |

### Differential Scanning Calorimeter (DSC) Characterizations

In some embodiments, Form M is characterized by a DSC curve having an endothermic signal (e.g., peak) at 143±20 °C (e.g., 143±10 °C (e.g., 143±5 °C (e.g., 143±4 °C (e.g., 143±3 °C (e.g., 143±2 °C (e.g., 143±1 °C (e.g., 143±0.5 °C))))))).

In some embodiments, Form M is characterized by a DSC curve having an endothermic signal (e.g., peak) at about 143 °C.

In some embodiments, Form M is characterized by a DSC curve having an endothermic signal (e.g., peak) associated with an enthalpy of about 6.1±4.0 J/g (e.g., 6.1±3.0 J/g (e.g., 6.1±2.0 J/g (e.g., 6.1±1.0 J/g (e.g., 6.1±0.5 J/g)))).

In some embodiments, Form M is characterized by a DSC curve having an endothermic signal (e.g., peak) associated with an enthalpy of about 6.1 J/g.

In some embodiments, Form M is characterized by a DSC curve having an endothermic signal (e.g., peak) at 153±20 °C (e.g., 153±10 °C (e.g., 153±5 °C (e.g., 153±4 °C (e.g., 153±3 °C (e.g., 153±2 °C (e.g., 153±1 °C (e.g., 153±0.5 °C))))))).

In some embodiments, Form M is characterized by a DSC curve having an endothermic signal (e.g., peak) at about 153 °C.

In some embodiments, Form M is characterized by a DSC curve having an endothermic signal (e.g., peak) associated with an enthalpy of about 9.7±4.0 J/g (e.g., 9.7±3.0 J/g (e.g., 9.7±2.0 J/g (e.g., 9.7±1.0 J/g (e.g., 9.7±0.5 J/g)))).

In some embodiments, Form M is characterized by a DSC curve having an endothermic signal (e.g., peak) associated with an enthalpy of about 9.7 J/g.

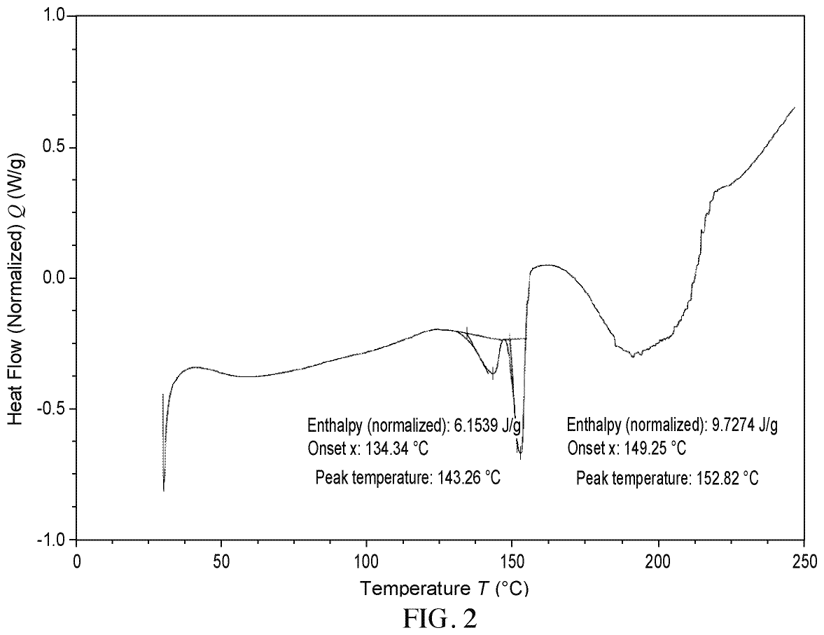

In some embodiments, Form M is characterized by a DSC curve substantially similar to that shown in FIG. 2.

### Thermogravimetric Analysis (TGA) Characterizations

In some embodiments, Form M shows a weight loss of from about 0.5% to about 10%, at a temperature ranging from about 34±20 °C (e.g., 34±10 °C (e.g., 34±5 °C (e.g., 34±4 °C (e.g., 34±3 °C (e.g., 34±2 °C (e.g., 34±1 °C (e.g., 34±0.5 °C))))))) to about 145±20 °C (e.g., 145±10 °C (e.g., 145±5 °C (e.g., 145±4 °C (e.g., 145±3 °C (e.g., 145±2 °C (e.g., 145±1 °C (e.g., 145±0.5 °C))))))), as measured by TGA.

In some embodiments, Form M shows a weight loss of about 4.7%, at a temperature ranging from about 34 °C to about 145 °C, as measured by TGA.

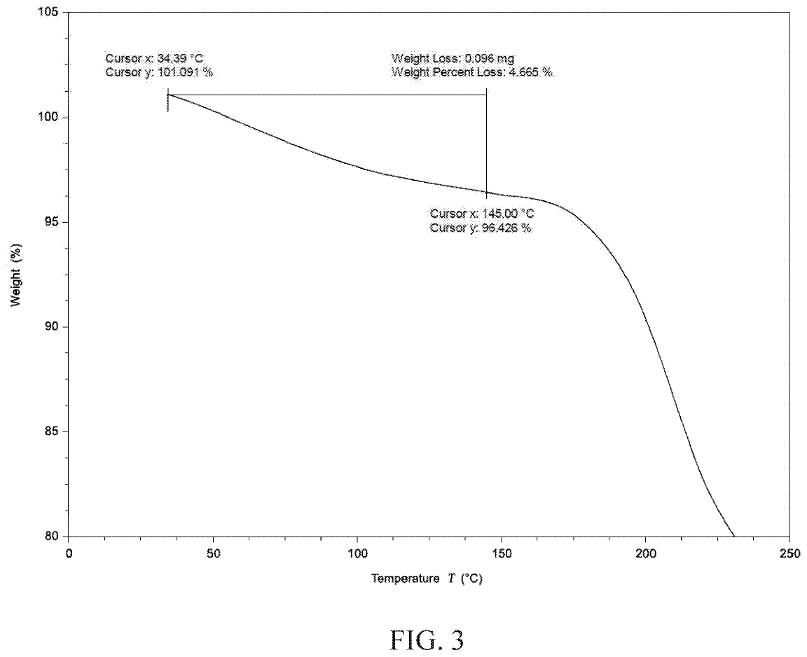

In some embodiments, Form M is characterized by a TGA profile substantially similar to that shown in FIG. 3.

### Methods of Preparing the Crystalline Form

In some aspects, the present disclosure features a method of preparing a morphic form (e.g., crystalline form) of a pharmaceutically acceptable salt of Compound No. 1, a solvate thereof, or a hydrate thereof.

In some aspects, the present disclosure provides a method of preparing a morphic form (e.g., crystalline form) of a pharmaceutically acceptable salt of Compound No. 1, a solvate thereof, or a hydrate thereof, comprising one or more steps as described herein.

In some aspects, the present disclosure provides a compound obtainable by, or obtained by, or directly obtained by a method for preparing a morphic form (e.g., crystalline form) of a pharmaceutically acceptable salt of Compound No. 1, a solvate thereof, or a hydrate thereof.

The morphic form (e.g., crystalline form) of a pharmaceutically acceptable salt of Compound No. 1, a solvate thereof, or a hydrate thereof can be prepared by any suitable technique known in the art. Particular processes for the preparation of these compounds are described further in the accompanying examples.

### Preparation of Form M

In some embodiments, the method comprises: (i) preparing a mixture comprising Compound No. 1, an acid, and a solvent, and (ii) removing the solvent from the mixture.

In some embodiments, the acid is malic acid.

In some embodiments, the acid is L-malic acid.

In some embodiments, the solvent is tetrahydrofuran (THF).

In some embodiments, the solvent is a mixture of methanol and water (e.g., at a ratio of about 95:5 v/v).

In some embodiments, the solvent is a mixture of methanol and water (e.g., at a ratio of about 98:2 v/v).

In some embodiments, the solvent is a mixture of methanol and water (e.g., at a ratio of about 10:1 v/v).

In some embodiments, the method further comprises equilibrating the mixture, e.g., at a temperature of about 5 °C or about 10 °C.

In some embodiments, the solvent is removed from the mixture at a temperature of about 35±15 °C, about 35±10 °C, about 35±9 °C, about 35±8 °C, about 35±7 °C, about 35±6 °C, about 35±5 °C, about 35±4 °C, about 35±3 °C, about 35±2 °C, or about 35±1 °C (e.g., about 35 °C).

### Pharmaceutical Compositions

In some aspects, the present disclosure features pharmaceutical compositions comprising a morphic form (e.g., crystalline form) of a pharmaceutically acceptable salt of Compound No. 1, a solvate thereof, or a hydrate thereof, and one or more pharmaceutically acceptable carriers or excipients.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It may be especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease and also preferably causing complete regression of the disease.

It is understood that the pharmaceutical compositions can be included in a container, pack, or dispenser together with instructions for administration.

### Methods of Use

In some aspects, the present disclosure provides a method of treating or preventing cancer in a subject, comprising administering to the subject a pharmaceutically effective amount of a morphic form (e.g., crystalline form) of a pharmaceutically acceptable salt of Compound No. 1, a solvate thereof, or a hydrate thereof.

In some aspects, the present disclosure provides a morphic form (e.g., crystalline form) of a pharmaceutically acceptable salt of Compound No. 1, a solvate thereof, or a hydrate thereof for use in treating or preventing cancer in a subject.

In some aspects, the present disclosure provides use of a morphic form (e.g., crystalline form) of a pharmaceutically acceptable salt of Compound No. 1, a solvate thereof, or a hydrate thereof, in the manufacture of a medicament for treating or preventing cancer in a subject.

In some embodiments, Form M of a pharmaceutically acceptable salt (e.g., an L-malate salt) of Compound No. 1 is administered.

### Suitable Subjects and Diseases

In some embodiments, the subject is a mammal.

In some embodiments, the subject is a human.

In some embodiments, the subject is a mouse.

In some embodiments, cancer is a solid tumor.

In some embodiments, the cancer is a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC), or any subtype thereof.

In some embodiments, the cancer is glioblastoma (GBM) or any subtype thereof. In some embodiments, the cancer is glioblastoma.

In some embodiments, the cancer is glioblastoma and the cancer is characterized by overexpression of EGFR.

In some embodiments, the cancer is methylated glioblastoma. In some embodiments, the cancer is unmethylated glioblastoma.

In some embodiments, the cancer is recurrent glioblastoma.

In some embodiments, the cancer is relapsed glioblastoma.

In some embodiments, the cancer is glioblastoma and the cancer, or a tumor or cell thereof, expresses at least one oncogenic variant of EGFR.

In some embodiments, the cancer is relapsed glioblastoma and the cancer, or a tumor or cell thereof, expresses at least one oncogenic variant of EGFR.

In some embodiments, the cancer is recurrent glioblastoma and the cancer, or a tumor or cell thereof, expresses at least one oncogenic variant of EGFR.

In some embodiments, the cancer is non-small cell lung cancer (NSCLC) or any subtype thereof.

In some embodiments, the cancer is non-small cell lung cancer (NSCLC).

In some embodiments, the cancer is recurrent non-small cell lung cancer (NSCLC).

In some embodiments, the cancer is relapsed non-small cell lung cancer (NSCLC).

In some embodiments, the cancer is NSCLC and the cancer, or a tumor or cell thereof, expresses at least one oncogenic variant of EGFR.

In some embodiments, the cancer is recurrent NSCLC and the cancer, or a tumor or cell thereof, expresses at least one oncogenic variant of EGFR.

In some embodiments, the cancer is relapsed NSCLC and the cancer, or a tumor or cell thereof, expresses at least one oncogenic variant of EGFR.

In some embodiments, the cancer is advanced or metastatic NSCLC.

In some embodiments, the cancer is advanced or metastatic NSCLC and the cancer, or a tumor or cell thereof, expresses at least one oncogenic variant of EGFR.

In some embodiments, the cancer is NSCLC, wherein the cancer has metastasized to the central nervous system (CNS).

In some embodiments, the cancer is advanced or metastatic NSCLC, wherein the cancer, or a tumor or cell thereof, expresses at least one oncogenic variant of EGFR, and wherein the cancer has metastasized to the central nervous system (CNS).

In some embodiments, the cancer is advanced or metastatic NSCLC, wherein the cancer, or a tumor or cell thereof, expresses at least one oncogenic variant of EGFR, and wherein the cancer has not metastasized to the central nervous system (CNS).

In some embodiments, the cancer is NSCLC, wherein the cancer has not metastasized to cerebrospinal fluid (CSF).

In some embodiments, the cancer is NSCLC, wherein the cancer has metastasized to cerebrospinal fluid (CSF).

In some embodiments, the cancer is glioblastoma, wherein the cancer has not metastasized to cerebrospinal fluid (CSF).

In some embodiments, the cancer is glioblastoma, wherein the cancer has metastasized to cerebrospinal fluid (CSF).

In some embodiments, the cancer is NSCLC, wherein the cancer has not metastasized to the brain.

In some embodiments, the cancer is NSCLC, wherein the cancer has metastasized to the brain.

In some embodiments, the subject has a central nervous system (CNS) disease.

In some embodiments, the subject does not have any CNS disease.

In some embodiments, the subject has a leptomeningeal disease.

In some embodiments, the subject does not have any leptomeningeal disease.

In some embodiments, the cancer is NSCLC and the subject has leptomeningeal disease.

In some embodiments, the cancer is glioblastoma and the subject has leptomeningeal disease.

In some embodiments, the cancer, or a tumor or a cell thereof, expresses an oncogenic variant of an ErbB receptor.

It is understood that an oncogenic variant of an ErbB receptor is an ErbB receptor protein that comprises at least one oncogenic mutation and that is produced as the result of the expression of a gene encoding the ErbB receptor that comprises at least one oncogenic mutation.

As would be appreciated by the skilled artisan, in the context of a gene (e.g. a gene encoding an ErbB receptor), an oncogenic mutation can include, but is not limited to a mutation that results in the substitution of one amino acid for another at a specific position within an ErbB receptor, a mutation that results in an insertion of one or more amino acids between two positions within an ErbB receptor, a mutation that results in the deletion of one more amino acids between two positions within an ErbB receptor, and mutation that results in a fusion of an ErbB receptor or portion thereof, with another protein, or portion thereof. As would be appreciated by the skilled artisan, in the context of a gene, an oncogenic mutation can include, but is not limited to, a missense mutation, a nonsynonymous mutation, an insertion of one or more nucleotides, a deletion of one or more nucleotides, an inversion and a deletion-insertion.

As would be appreciated by the skilled artisan, in the context of a protein (e.g. an ErbB receptor), an oncogenic mutation can include, but is not limited to, the substitution of one amino acid for another at a specific position within an ErbB receptor, an insertion of one or more amino acids between two positions within an ErbB receptor, a deletion of one more amino acids between two positions within an ErbB receptor, and a fusion of an ErbB receptor, or portion thereof, with another protein, or portion thereof.

In some embodiments, the oncogenic variant of the ErbB receptor comprises an allosteric mutation.

In some embodiments, the oncogenic variant of an ErbB receptor is an allosteric variant of the ErbB receptor.

In some embodiments, the ErbB receptor is an epidermal growth factor receptor (EGFR) or a human epidermal growth factor receptor 2 (HER2) receptor.

In some embodiments, the ErbB receptor is an epidermal growth factor receptor (EGFR).

In some embodiments, the ErbB receptor is a HER2 receptor.

In some embodiments, the ErbB receptor is a HER3 receptor.

In some embodiments, the ErbB receptor is a HER4 receptor.

In some embodiments, the cancer, or a tumor or a cell thereof, expresses an oncogenic variant of an epidermal growth factor receptor (EGFR).

In some embodiments, the oncogenic variant of EGFR is an allosteric variant of EGFR.

In some embodiments, the oncogenic variant of EGFR comprises an allosteric mutation.

In some embodiments, the cancer, or a tumor or a cell thereof, expresses an oncogenic variant of a HER2 receptor.

In some embodiments, the oncogenic variant of the HER2 receptor is an allosteric variant of the HER2 receptor.

In some embodiments, the oncogenic variant of the HER2 receptor comprises an allosteric mutation.

In some embodiments, the oncogenic variant of an EGFR comprises an EGFR variant III (EGFR-Viii) mutation.

In some embodiments, the oncogenic variant of EGFR comprises an EGFR variant II (EGFR-Vii) mutation.

In some embodiments, the oncogenic variant of EGFR comprises an EGFR variant VI (EGFR-Vvi) mutation.

### DEFINITIONS

It is understood that the compounds described herein include the compounds themselves, as well as their salts, and their solvates, if applicable. A salt, for example, can be formed between an anion and a positively charged group (e.g., amino) on a substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

Unless explicitly indicated otherwise, the terms “approximately” and “about” are synonymous. In some embodiments, “approximately” and “about” refer to the recited amount, value, dose, or duration±20%, ±15%, ±10%, ±8%, ±6%, ±5%, ±4%, ±2%, ±1%, or 0.5%. In some embodiments, “approximately” and “about” refer to the listed amount or duration±10%, ±8%, ±6%, ±5%, ±4%, or ±2%. In some embodiments, “approximately” and “about” refer to the listed amount, value, dose, or duration±5%. In some embodiments, “approximately” and “about” refer to the listed amount, value, dose, or duration±2%. In some embodiments, “approximately” and “about” refer to the listed amount, value, dose, or duration±1%. In some embodiments, when referring to the value of an X-ray diffraction (“XRPD”) signal (e.g., peak), “approximately” and “about” refer to the listed value±0.2, ±0.15, ±0.1, ±0.05, ±0.02, or ±0.01.

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted benzene compound. Suitable cations include sodium ion, potassium ion, magnesium ion, calcium ion, and an ammonium cation such as tetramethylammonium ion. The substituted benzene compounds also include those salts containing quaternary nitrogen atoms.

It is understood that the compounds of the present disclosure, for example, the salts of the compounds, can exist in either hydrated or unhydrated (the anhydrous) form or as solvates with other solvent molecules. Nonlimiting examples of hydrates include monohydrates and dihydrates. Nonlimiting examples of solvates include ethanol solvates and acetone solvates.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art.

It is to be understood that, unless otherwise stated, any description of a method of treatment or prevention includes use of a crystalline form of Compound No. 1 or a pharmaceutically acceptable salt thereof to provide such treatment or prevention as is described herein. It is to be further understood, unless otherwise stated, any description of a method of treatment or prevention includes use of a crystalline form of Compound No. 1 or a pharmaceutically acceptable salt thereof to prepare a medicament to treat or prevent such condition. The treatment or prevention includes treatment or prevention of human or non-human animals including rodents and other disease models.

It is to be understood that, unless otherwise stated, any description of a method of treatment includes use of a crystalline form of Compound No. 1 or a pharmaceutically acceptable salt thereof to provide such treatment as is described herein. It is to be further understood, unless otherwise stated, any description of a method of treatment includes use of a crystalline form of Compound No. 1 or a pharmaceutically acceptable salt thereof to prepare a medicament to treat such condition. The treatment includes treatment of human or non-human animals including rodents and other disease models.

As used herein, the term “subject” refers to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

In some embodiments, the term “subject in need thereof” can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject in need thereof may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a crystalline form of Compound No. 1 or a pharmaceutically acceptable salt thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that “solubility” or “solubility rating” refers to the property of a polymorph (e.g., Form M) disclosed herein to dissolve in a liquid solvent and form a homogeneous solution. In some embodiments, solubility is expressed as a concentration, either by mass of solute per unit volume of solvent (e.g., g of solute per kg of solvent, g per dL (100 mL), mg/ml, etc.), molarity, molality, mole fraction, or other similar descriptions of concentration. A person of skill in the art may understand that the maximum equilibrium amount of solute that can dissolve per amount of solvent is the solubility of that solute in that solvent under the specified conditions, including temperature, pressure, pH, and the nature of the solvent.

As used herein, “stable” refers to a polymorph that maintains purity, appearance, and/or analytical parameters over a defined time and temperature as compared to the polymorph as isolated. In some embodiments, the “stable” polymorph exhibits less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity over a set period of time (e.g., 1 day, 2 days, 3 days, 4 days, 5 days, 6 days, one week, two weeks, three weeks, one month, two months, three months, or four months).

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. In some embodiments, the pharmaceutically acceptable salt of a compound is also a prodrug of the compound. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It is understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### Exemplary Embodiments

Exemplary Embodiment No. 1. A morphic form of a pharmaceutically acceptable salt of Compound No. 1:

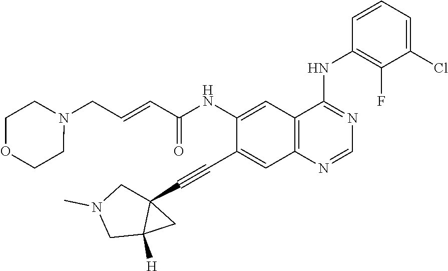 [SMILES: CN1C[C@H]2C[C@@]2(C#Cc2cc3ncnc(Nc4cccc(Cl)c4F)c3cc2NC(=O)/C=C/CN2CCOCC2)C1]

a solvate thereof, or a hydrate thereof.

Exemplary Embodiment No. 2. The morphic form of Exemplary Embodiment No. 1, wherein the morphic form is a crystalline form.

Exemplary Embodiment No. 3. The morphic form of any one of the preceding Exemplary Embodiments, wherein the morphic form is Form M of a pharmaceutically acceptable salt of Compound No. 1, a solvate thereof, or a hydrate thereof.

Exemplary Embodiment No. 4. The morphic form of any one of the preceding Exemplary Embodiments, wherein Form M is characterized by an X-ray powder diffraction (“XRPD”) pattern comprising signals at 4.5±0.2, 8.9±0.2, and 21.7±0.2° 2θ using Cu Kα radiation.

Exemplary Embodiment No. 5. The morphic form of any one of the preceding Exemplary Embodiments, wherein the XRPD pattern of Form M further comprises at least one signal selected from 13.3±0.2, 13.9±0.2, and 19.7±0.2° 2θ using Cu Kα radiation.

Exemplary Embodiment No. 6. The morphic form of any one of the preceding Exemplary Embodiments, wherein the XRPD pattern of Form M further comprises at least one signal selected from 17.9±0.2, 18.9±0.2, 22.2±0.2, and 26.7±0.2° 2θ using Cu Kα radiation.

Exemplary Embodiment No. 7. The morphic form of any one of the preceding Exemplary Embodiments, wherein Form M is characterized by an XRPD pattern substantially similar to that shown in FIG. 1.

Exemplary Embodiment No. 8. The morphic form of any one of the preceding Exemplary Embodiments, wherein Form M is characterized by an XRPD pattern comprising one or more of the signals (e.g., peaks) at approximately the positions shown in Table A.

Exemplary Embodiment No. 9. The morphic form of any one of the preceding Exemplary Embodiments, wherein Form M is characterized by a DSC profile having an endothermic signal at 143±20 °C.

Exemplary Embodiment No. 10. The morphic form of any one of the preceding Exemplary Embodiments, wherein Form M is characterized by a DSC profile having an endothermic signal at 153±20 °C.

Exemplary Embodiment No. 11. The morphic form of any one of the preceding Exemplary Embodiments, wherein Form M shows a weight loss of from about 0.5% to about 10%, at a temperature ranging from about 34±20 °C to about 145±20 °C, as measured by TGA.

Exemplary Embodiment No. 12. A pharmaceutical composition comprising a therapeutically effective amount of the morphic forms of any one of the preceding Exemplary Embodiments and a pharmaceutically acceptable excipient.

Exemplary Embodiment No. 13. A method of inhibiting an oncogenic variant of an ErbB receptor, comprising administering to the subject in need thereof a therapeutically effective amount of the morphic form, or pharmaceutical composition, of any one of the preceding Exemplary Embodiments.

Exemplary Embodiment No. 14. A method of preventing or treating cancer, comprising administering to the subject in need thereof a therapeutically effective amount of the morphic form, or pharmaceutical composition, of any one of the preceding Exemplary Embodiments.

Exemplary Embodiment No. 15. A method of preventing or treating cancer, comprising: i) identifying a subject candidate as the subject in need of the treatment when that at least one oncogenic variant of an ErbB receptor is present in the subject, or in a biological sample from the subject; and ii) administering to the subject in need of the treatment a therapeutically effective amount of the morphic form, or pharmaceutical composition, of any one of the preceding Exemplary Embodiments.

Exemplary Embodiment No. 16. A method of preventing or treating cancer, comprising administering to the subject in need thereof the morphic form, or pharmaceutical composition, of any one of the preceding Exemplary Embodiments when that at least one oncogenic variant of an ErbB receptor is identified as being present in the subject, or in a biological sample from the subject.

Exemplary Embodiment No. 17. The morphic form, or pharmaceutical composition, of any one of the preceding Exemplary Embodiments for use in the inhibition of an oncogenic variant of an ErbB receptor.

Exemplary Embodiment No. 18. The morphic form, or pharmaceutical composition, of any one of the preceding Exemplary Embodiments for use in the prevention or treatment of cancer.

Exemplary Embodiment No. 19. The morphic form, or pharmaceutical composition, of any one of the preceding Exemplary Embodiments for use in the prevention or treatment of cancer in a subject, wherein at least one oncogenic variant of an ErbB receptor is present in the subject, or in a biological sample from the subject.

Exemplary Embodiment No. 20. Use of the morphic form of any one of the preceding Exemplary Embodiments in the manufacture of a medicament for inhibiting an oncogenic variant of an ErbB receptor.

Exemplary Embodiment No. 21. Use of the morphic form of any one of the preceding Exemplary Embodiments in the manufacture of a medicament for preventing or treating cancer.

Exemplary Embodiment No. 22. The morphic form, pharmaceutical composition, method, or use of any one of the preceding Exemplary Embodiments, wherein the cancer is a solid tumor, a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC), or any subtype thereof.

Exemplary Embodiment No. 23. The morphic form, pharmaceutical composition, method, or use of any one of the preceding Exemplary Embodiments, wherein the cancer or a tumor or a cell thereof expresses an oncogenic variant of an epidermal growth factor receptor (EGFR).

Exemplary Embodiment No. 24. The morphic form, pharmaceutical composition, method, or use of any one of the preceding Exemplary Embodiments, wherein the oncogenic variant is an oncogenic variant in an ErbB receptor, epidermal growth factor receptor (EGFR), of a HER2 receptor, or a HER-4 receptor and wherein the oncogenic variant in the ErbB receptor, EGFR, or HER2 receptor is an allosteric variant.

Exemplary Embodiment No. 25. The morphic form, pharmaceutical composition, method, or use of any one of the preceding Exemplary Embodiments, wherein the subject or the cancer is insensitive or resistant to treatment with one or more of gefinitinib, erlotinib, afatinib, osimertinib, and necitunumab.

Exemplary Embodiment No. 26. The morphic form, pharmaceutical composition, method, or use of any one of the preceding Exemplary Embodiments, wherein the sequence encoding the oncogenic variant of the EGFR comprises a deletion of exon 20 or a portion thereof and wherein the cancer, tumor or cell thereof does not comprise an oncogenic variation in a sequence encoding one or more of an EGFR kinase domain (KD), BRAF, NTRK, and KRAS or a marker indicating responsiveness to immunotherapy.

Exemplary Embodiment No. 27. The morphic form, pharmaceutical composition, method, or use of any one of the preceding Exemplary Embodiments, wherein the oncogenic variant or the oncogenic mutation is detected by a Food and Drug Administration (FDA)-approved diagnosis.

Exemplary Embodiment No. 28. The morphic form, pharmaceutical composition, method, or use of any one of the preceding Exemplary Embodiments, wherein the subject has an adverse reaction to treatment with a Type I inhibitor.

Exemplary Embodiment No. 29. The morphic form, pharmaceutical composition, method, or use of any one of the preceding Exemplary Embodiments, wherein the subject has an adverse reaction to treatment with one or more of gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291.

### Examples

It is understood that the experimental values described in the present application are approximate and subject to instrumental variations.

X-ray Powder Diffractometer (XRPD): XRPD analysis was performed using a Bruker D8 Advance X-ray powder diffractometer. Analysis was performed using parameters as set forth below.

XRPD Method

| | |
|---|---|
| Detector | LYNXEYE_XE_T(1D mode) |
| Open angle | 2.94° |
| Scan mode | Continuous PSD fast |
| Radiation | Cu/K-Alpha1 (λ = 1.5418 Å) |
| X-ray generator power | 40 kV, 40 mA |
| Step size | 0.02° |
| Time per step | 0.12 second per step |
| Scan range | 3° to 40° |
| Primary beam path | Twin_Primary motorized slit 10.0 |
| slits | mm by sample length; |
| | SollerMount axial soller 2.5° |
| Secondary beam path | Detector OpticsMount soller slit 2.5°; |
| slits | Twin_Secondary motorized slit 5.2 mm |
| Sample rotation speed | 15 rpm |

Differential Scanning Calorimetric (DSC): DSC analysis was performed using TA Discover 2500 or Q2000. Analysis was performed using parameters as set forth below.

| Sample pan | Tzero pan and Tzero hermetic lid with a pin hole | |
|---|---|---|
| Temperature range | 30 to 250 °C or before decomposition | |
| Heating rate | 10 | °C/min |
| Nitrogen flow | 50 | mL/min |
| Sample mass | ~1-2 | mg |

Thermal Gravimetric Analysis (TGA): TGA analysis was performed using Discover 5500. Analysis was performed using parameters as set forth below.

| Sample pan | Aluminum, open | |
|---|---|---|
| Nitrogen flow | Balance 10 mL/min; sample 25 mL/min | |
| Start | Ambient condition (below 35 °C) | |
| temperature | | |
| Final | 300 °C or abort next segment if weight <80% (w/w) | |
| temperature | (The weight loss of the compound | |
| | is no more than 20% (w/w) | |
| Heating rate | 10 | °C/min |
| Sample mass | ~2-10 | mg |

Dynamic Vapor Sorption (DVS): DVS analysis was performed using Intrinsic. Analysis was performed using parameters as set forth below.

| Instrument | Intrinsic | |
|---|---|---|
| Total gas flow | 200 | sccm |
| Oven temperature | 25 | °C |

| | |
|---|---|
| Solvent | Water |
| Method | Cycle: 40-95-0-95-40% RH |
| | Stage Step: 10% |
| | Equilibrium: 0.002 dm/dt (%/min) |
| | Minimum dm/dt stability duration: 60 min |
| | Maximum dm/dt stage time: 360 min |

Nuclear Magnetic Resonance (NMR): NMR analysis was performed using Bruker Avance-AV 400M at a frequency of 400 MHz, a 5 mm PABBO BB-1H/D probe, 8 scans, a temperature of 297.6 K, and a relaxation delay of 1 second.

High Performance Liquid Chromatograph (HPLC): HPLC analysis was performed using SHIMADZU LC-20AD/Agilent 1260 infinity II Binary Pump. Analysis was performed using parameters as set forth below.

| HPLC method | Wavelength: 272 nm | | |
|---|---|---|---|
| | Column: PDS-HPLC-010 | | |
| | Detector: DAD | | |
| | Column temperature: 50°C | | |
| | Flow rate: 1.0 mL/min | | |
| | Mobile phase A: 375 μL TFA in 1 L water | | |
| | Mobile phase B: 187.5 μL TFA in 1 L ACN | | |
| | Injection volume: 5 μL | | |
| | Gradient: | | |
| | Time (min) | Mobile Phase A (%) | Mobile Phase B (%) |
| | 0.01 | 95 | 5 |
| | 1.0 | 95 | 5 |
| | 6.0 | 20 | 80 |
| | 8.0 | 20 | 80 |
| | 8.1 | 95 | 5 |
| | 9.9 | 95 | 5 |
| | 10.0 | | stop |

### Example 1. Exemplary Preparation of Crystalline Form M

About 250 mg of L-malic acid (about 2.0 equivalent) was weighed into a 40 mL glass bottle. 2 mL of THF was added to dissolve L-malic acid. This clear solution was stirred at 25 °C with a rate of 250 rpm. About 500 mg of Compound No. 1 was weighed into an 8 mL glass bottle and 4 mL of THF was added to dissolve the free base. Then, 200 μL of Compound No. 1 solution was dropped into L-Malic acid THF solution. An oil-like material was obtained at first. Meanwhile, about 5 mg of the L-malate salt seeds was added into the 40 mL bottle. A suspension was obtained. The rest of the free base solution was added into the suspension slowly. Another 1.5 mL of THF was added into the suspension after massive solids precipitated out. This suspension was stirred at 25 °C with a rate of 400 rpm for 3 days. The obtained suspension was taken out and the solids were separated out by centrifugation. The solid part was dried under vacuum at 30 °C for 3 h. About 700 mg of the L-malate salt Form M was obtained as orange solids in a yield of about 90%. The dry cake was characterized by XRPD and 1H-NMR. For this dry cake, about 2.8 equiv. L-malic acid was detected by 1H-NMR.

To explore further optimization of the crystallinity of the salt, about 25 mg of dry cake were re-equilibrated in 7 different solvents for 3 days. Solids obtained were analyzed by XRPD and 1H-NMR. Solid part was dried under vacuum at 35 °C for 2 h. Results were summarized in Table 1. After re-equilibration in methanol/water (v:v=95:5), crystallinity of the L-malate salt improved and it showed reasonable stoichiometry. For the L-malate salt Form M, its crystallinity decreased after being dried under vacuum at 50 °C for 2 h.

TABLE 1
Preparation of Form M: Re-slurry Experiment Results.

| Solvent | Comments | Dry cakes |
|---|---|---|
| MeOH | High crystallinity | Crystallinity |
| | 2.1 equiv. L-malic acid | decreased |
| Acetone | Medium crystallinity | |
| | 2.8 equiv. L-malic acid | |
| ACN | Medium crystallinity | |
| | 2.5 equiv. L-malic acid | |
| MTBE | Medium crystallinity | |
| | 3.0 equiv. L-malic acid | |
| DCM | Medium crystallinity | |
| | 2.9 equiv. L-malic acid | |
| MeOH:H₂O | High crystallinity | Crystallinity |
| (v:v = 95:5) | 1.8 equiv. L-malic acid | decreased |
| THF | Medium crystallinity | |
| | 2.4 equiv. L-malic acid | |

The solvent system of MeOH and H₂O was selected for optimizing the crystallization. About 100 μL of the suspension in methanol/water (v:v=98:2) system was added into a 2 mL glass vial. More water was added to adjust solvent system to methanol/water (v:v=95:5). This suspension was stirred at 10 °C for 2 days. The suspension was taken out and centrifuged at 10 °C. The solids were characterized by XRPD. Crystallinity of the L-malate salt improved and its stoichiometry was more reasonable (about 2.2 equiv. L-malic acid).

To further optimize the stoichiometry of this L-malate salt, more water was added to adjust solvent system to methanol/water (v:v=10:1). The obtained suspension was stirred at 10 °C for 4 days. The obtained suspension was taken out and the orange solids were separated out by centrifugation. The solid part was dried at 25 °C/50% RH for 16 h. About 180 mg of the L-malate salt Form M was obtained as brown solids in a yield of about 50%. Its stoichiometry of free form to L-malic acid is 1:2.1.

### Example 2. Studies of Properties of Form M

Bulk Stability: Form M was evaluated for bulk stability studies. The bulk stability study was conducted in 3 conditions including 25 °C/92% RH in an open container, 40 °C/75% RH in an open container and 60 °C in a tight container for 1 week, 2 weeks and 4 weeks, respectively. Table 2 summarizes observation of the stability study.

TABLE 2
Form M

| Parameter | |
|---|---|
| Initial Purity by | 94.0% |
| HPLC | |

| | |
|---|---|
| Purity | Color |

Solid state, 25 °C/92% RH, open container, 1 week

| | | |
|---|---|---|
| Bulk (HPLC) | 95.1% | No change of color |

| | |
|---|---|
| Bulk (XRPD) | No form change (Agglomeration) |

Solid state, 40 °C/75% RH, open container, 1 week

| | | |
|---|---|---|
| Bulk (HPLC) | 92.5% | No change of color |

| | |
|---|---|
| Bulk (XRPD) | No form change |

Solid state, 60 °C, tight container, 1 week

| | | |
|---|---|---|
| Bulk (HPLC) | 63.2% | No change of color |

| | |
|---|---|
| Bulk (XRPD) | No form change (Crystallinity decreased) |

Solid state, 25 °C/92% RH, open container, 2 weeks

| | | |
|---|---|---|
| Bulk (HPLC) | 95.2% | No change of color |

| | |
|---|---|
| Bulk (XRPD) | Sticky sample; |
| | No form change after dried under ambient |
| | condition |

Solid state, 40 °C/75% RH, open container, 2 weeks

| | | |
|---|---|---|
| Bulk (HPLC) | 90.3% | No change of color |

| | |
|---|---|
| Bulk (XRPD) | No form change |

Solid state, 60 °C, tight container, 2 weeks

| | | |
|---|---|---|
| Bulk (HPLC) | 50.5% | No change of color |

| | |
|---|---|
| Bulk (XRPD) | No form change (Crystallinity decreased) |

Solid state, 25 °C/92% RH, open container, 4 weeks

| | | |
|---|---|---|
| Bulk (HPLC) | 95.7% | No change of color |

| | |
|---|---|
| Bulk (XRPD) | Sticky sample; |
| | No form change after dried under ambient |
| | condition |

Solid state, 40 °C/75% RH, open container, 4 weeks

| | | |
|---|---|---|
| Bulk (HPLC) | 86.3% | No change of color |

| | |
|---|---|
| Bulk (XRPD) | No form change |

Solid state, 60 °C, tight container, 4 weeks

| | | |
|---|---|---|
| Bulk (HPLC) | 43.6% | No change of color |

| | |
|---|---|
| Bulk (XRPD) | No form change |
| | (Crystallinity decreased) |

Solubility: 6.39 mg of the L-malate salt Form M (equal to 4 mg free base) was weighed into an 8 mL vial. 2 mL of aqueous media was added, respectively. The suspension was stirred at 37 °C with 400 rpm for 2 h and 24 h. The suspension was taken out after 2 h and 24 h and then centrifuged at 14,000 rpm for 5 min. The supernatant was analyzed by HPLC. The pH of the supernatant was tested. The solids obtained (wet cakes) were characterized by XRPD. Table 3 summarizes the observation of the solubility study.

TABLE 3

| | Form M | | | |
|---|---|---|---|---|
| | 2 h | | 24 h | |
| | Solubility | XRPD | Solubility | XRPD |
| pH 1.0 | >2 mg/mL | Solids were not | >2 mg/mL | Solids were not |
| (0.1N HCl) | pH 0.9 | enough for XRPD | pH 0.8 | enough for XRPD |
| | | analysis | | analysis |
| pH 4.5 | >2 mg/mL | Solids were not | >2 mg/mL | Solids were not |
| (50 mM) | pH 4.5* | enough for XRPD | pH 4.4 | enough for XRPD |
| acetate | | analysis | | analysis |
| buffer | | | | |
| pH 6.8 | 182.9 μg/mL | Dissociated to | 188.7 μg/mL | Dissociated to |
| (50 mM) | pH 6.2 | free base | pH 6.3 | free base |
| phosphate | | | | |
| buffer | | | | |
| FeSSIF-v1, | Degradation | Solids were not | Degradation | Solids were not |
| pH 5.0 | pH 4.9 | enough for XRPD | pH 4.8 | enough for XRPD |
| | | analysis | | analysis |
| FaSSIF-v1, | Degradation | Solids were not | Degradation | Solids were not |
| pH 6.5 | pH 4.9 | enough for XRPD | pH 4.8 | enough for XRPD |
| | | analysis | | analysis |
| SGF, | >2 mg/mL | Solids were not | >2 mg/mL | Solids were not |
| pH 2.0 | pH 2.8 | enough for XRPD | pH 2.6 | enough for XRPD |
| | | analysis | | analysis |
| Water | 1488.0 μg/mL | Solids were not | 1779.2 μg/mL | Solids were not |
| | pH 4.3 | enough for XRPD | pH 4.3 | enough for XRPD |
| | | analysis | | analysis |

Hygroscopicity: The hygroscopicity for Form M was examined by DVS. Table 4 summarizes the observation of the hygroscopicity study.

TABLE 4
Hygroscopicity by DVS at 25 °C dm/dt = 0.002%

| | |
|---|---|
| | L-malate salt Form M |
| DVS | 1.5% at 70% RH |
| | 37.1% at 95% RH |

| Batch/ | Sorp. | Desorp. | Sorp. | Desorp. |
|---|---|---|---|---|
| Sample ID | (%) | (%) | (%) | (%) |
| 0% | | 0.0 | 0.0 | |
| 10% | | 1.4 | 0.6 | |
| 20% | | 2.5 | 1.4 | |
| 30% | | 3.6 | 2.2 | |
| 40% | 4.1 | 5.0 | 3.1 | 5.2 |
| 50% | 4.4 | 6.1 | 4.2 | 6.4 |
| 60% | 4.9 | 7.4 | 5.6 | 7.4 |
| 70% | 5.6 | 9.3 | 7.5 | 8.7 |
| 80% | 6.9 | 13.5 | 9.7 | 11.4 |
| 90% | 15.3 | 29.5 | 15.8 | 20.9 |
| 95% | 41.2 | 41.2 | 31.5 | 31.5 |

| | |
|---|---|
| XRPD after DVS test | No form change |

### EQUIVALENTS

It is understood that the invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A morphic form of a pharmaceutically acceptable salt of Compound No. 1: 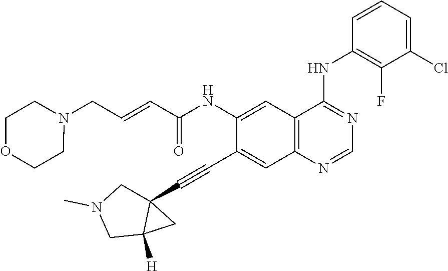 [SMILES: CN1C[C@H]2C[C@@]2(C#Cc2cc3ncnc(Nc4cccc(Cl)c4F)c3cc2NC(=O)/C=C/CN2CCOCC2)C1]
a solvate thereof, or a hydrate thereof.

2. The morphic form of claim 1, wherein the morphic form is a crystalline form.

3. The morphic form of any one of the preceding claims, wherein the morphic form is Form M of a pharmaceutically acceptable salt of Compound No. 1, a solvate thereof, or a hydrate thereof.

4. The morphic form of any one of the preceding claims, wherein Form M is characterized by an X-ray powder diffraction (“XRPD”) pattern comprising signals at 4.5±0.2, 8.9±0.2, and 21.7±0.2° 2θ using Cu Kα radiation.

5. The morphic form of any one of the preceding claims, wherein the XRPD pattern of Form M further comprises at least one signal selected from 13.3±0.2, 13.9±0.2, and 19.7±0.2° 2θ using Cu Kα radiation.

6. The morphic form of any one of the preceding claims, wherein the XRPD pattern of Form M further comprises at least one signal selected from 17.9±0.2, 18.9±0.2, 22.2±0.2, and 26.7±0.2° 2θ using Cu Kα radiation.

7. The morphic form of any one of the preceding claims, wherein Form M is characterized by an XRPD pattern substantially similar to that shown in FIG. 1.

8. The morphic form of any one of the preceding claims, wherein Form M is characterized by an XRPD pattern comprising one or more of the signals (e.g., peaks) at approximately the positions shown in Table A.

9. The morphic form of any one of the preceding claims, wherein Form M is characterized by a DSC profile having an endothermic signal at 143±20 °C.

10. The morphic form of any one of the preceding claims, wherein Form M is characterized by a DSC profile having an endothermic signal at 153±20 °C.

11. The morphic form of any one of the preceding claims, wherein Form M shows a weight loss of from about 0.5% to about 10%, at a temperature ranging from about 34±20 °C to about 145±20 °C, as measured by TGA.

12. A pharmaceutical composition comprising a therapeutically effective amount of the morphic forms of any one of the preceding claims and a pharmaceutically acceptable excipient.

13. A method of preventing or treating cancer, comprising administering to the subject in need thereof a therapeutically effective amount of the morphic form, or pharmaceutical composition, of any one of the preceding claims.

14. The morphic form, or pharmaceutical composition of any one of the preceding claims for use in the prevention or treatment of cancer.

15. Use of the morphic form of any one of the preceding claims in the manufacture of a medicament for preventing or treating cancer.

16. The morphic form, pharmaceutical composition, method, or use of any one of the preceding claims, wherein the cancer is a solid tumor, a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC), or any subtype thereof.

17. The morphic form, pharmaceutical composition, method, or use of any one of the preceding claims, wherein the oncogenic variant is an oncogenic variant in an ErbB receptor, epidermal growth factor receptor (EGFR), of a HER2 receptor, or a HER-4 receptor and wherein the oncogenic variant in the ErbB receptor, EGFR, or HER2 receptor is an allosteric variant.

18. The morphic form, pharmaceutical composition, method, or use of any one of the preceding claims, wherein the subject or the cancer is insensitive or resistant to treatment with one or more of gefinitinib, erlotinib, afatinib, osimertinib, and necitunumab.

19. The morphic form, pharmaceutical composition, method, or use of any one of the preceding claims, wherein the sequence encoding the oncogenic variant of the EGFR comprises a deletion of exon 20 or a portion thereof and wherein the cancer, tumor or cell thereof does not comprise an oncogenic variation in a sequence encoding one or more of an EGFR kinase domain (KD), BRAF, NTRK, and KRAS or a marker indicating responsiveness to immunotherapy.

20. The morphic form, pharmaceutical composition, method, or use of any one of the preceding claims, wherein the subject has an adverse reaction to treatment with a Type I inhibitor.

21. The morphic form, pharmaceutical composition, method, or use of any one of the preceding claims, wherein the subject has an adverse reaction to treatment with one or more of gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291.
